(19) United States
(12) Patent Application Publication
(10) Pub. No.: US 2024/0172980 A1
(43) Pub. Date: May 30, 2024

(54) METHOD AND PROCESSING UNIT FOR IDENTIFYING STATE OF MIND OF A USER BY ANALYZING DRAWINGS DRAWN BY A USER

(71) Applicant: KASEN-KELLS CONSULTING, LLC d/b/a COLOR MENDING, Austin, TX (US)

(72) Inventor: Chelsea R. Kasen-Kells, Austin, TX (US)

(21) Appl. No.: 18/525,189

(22) Filed: Nov. 30, 2023

Related U.S. Application Data
(60) Provisional application No. 63/429,087, filed on Nov. 30, 2022.

(51) Int. Cl. A61B 5/16; G06F 3/04883; G06V 10/20; G16H 50/20

(52) U.S. Cl. CPC A61B 5/165; G06F 3/04883; G06V 10/255; G16H 50/20

(57) ABSTRACT

Method and processing unit for identifying the state of mind of a user are described. The method for identifying state of mind of the user includes facilitating the user to provide a drawing drawn by the user. Further, the method includes receiving the provided drawing and user data while providing the image and converting the received drawing into a machine understandable format. The method further includes pre-processing the received drawing to identify one or more shapes and/or one or more colors in the received drawing. Thereafter, the method includes identifying the state of mind of the user based on the received user data, the identified one or more shapes, and/or the identified one or more colors by employing one or more Machine Learning (ML) models.

The present application claims the benefit of U.S. Provisional Application No. 63/429,087, filed Nov. 30, 2022; all of which is incorporated herein by reference in its entirety.

### FIELD OF THE PRESENT DISCLOSURE

Embodiments of the present disclosure generally relate to identifying the state of mind of a user. In particular, embodiments of the present disclosure relate to a method and processing unit for identifying the state of mind of a user by analyzing drawings drawn by the user.

### BACKGROUND OF THE DISCLOSURE

Accurate identification of state of mind of a user may be essential to decide which therapy would be apt for the user. Conventionally, a psychiatrist or a therapist may have a conversation or monitor the user to determine the state of mind of the user. In some cases, such conversations and monitoring may not be enough to accurately determine the state of mind. Currently, there may be several applications and systems which automatically determine the state of mind of the user without a need for the psychiatrist or the therapist. Some of the applications teach to determine the state of mind using a pre-defined questionnaire to interrogate the user. Answers provided by the user may be analyzed by a computing system to determine the state of mind. In some applications, images, and video of user's expressions and real-time behaviors may be captured and analyzed by the computing system to determine the state of mind. Using the determined state of mind, therapy sessions for the user may be decided and provided.

U.S. Pat. No. 9,839,762B2 teaches assessing an individual's psychological status by subjecting an individual to a treatment-determining test. The treatment-determining test includes the administration of a questionnaire containing a plurality of pre-determined questions designed to assess the individual's emotional state. Data obtained from the treatment-determining test is electronically analyzed to determine the treatment session to be administered to the individual. In the patent, the treatment session may include a specific sequence of said colored light, said one or more colored images, said auditory notes, or said one or more aromas.

However, in such ways of determining the state of mind, there are chances of manipulating the answers that are intended to be provided by the user. The user may be conscious when providing the answers or when the video or image of the user is captured. Thus, accurate detection of the state of mind or behavior characteristics may not be achieved using such techniques.

Further, in a non-patent literature titled “The art of mandala: Akshita Gandhi”, it is taught that a mandala technique is used to determine the state of mind. The mandala is an abstract design made of intricate geometric compositions created on the base of a circle. A user is provisioned to choose colors to create art inside the mandala. Each color represents mood, inner feelings, and thought, and it reflects the state of mind and being of the user. However, it is not disclosed in the literature how the created art is used to determine the state of mind. Conventionally, a mentor or a therapist may review the art manually to analyze and understand the state of mind of the user.

Therefore, there is a requirement for a method and processing unit for identifying the state of mind of the user that overcomes the drawbacks of the existing technologies.

The information disclosed in this background of the disclosure section is only for enhancement of understanding of the general background of the disclosure and should not be taken as an acknowledgment or any form of suggestion that this information forms existing information already known to a person skilled in the art.

### BRIEF SUMMARY OF THE DISCLOSURE

The subject disclosure is broadly related to identifying the state of mind of a user. The subject disclosure teaches to develop a model which may analyze image drawn on a user device and perform interpretation that implies the user's behavior analysis. For identifying the state of mind, two parameters i.e., shapes and colors in the image drawn by the user are analyzed in real-time to give interpretation or decide the behavior characteristics of the user. The developed model works as an emotion AI (Artificial Intelligence) model or artificial emotional intelligence model which is trained to recognize an individual's feelings or emotions behind a picture drawn and respond in written text. In other words, the model allows computers to generate emotional responses to visual art and justify those emotions in language. The algorithm categorizes the user's drawing into a set of parameters based on shapes and colors and then explains in written text what it is in the image that justifies the emotional read, basically deciphering emotions within a given shape or drawing. The interpretation results accurately reflect the abstract content of the drawing in ways that go well beyond the capabilities of existing computer vision algorithms derived from documentary photographic datasets.

An embodiment of the present disclosure includes a processing unit for identifying a state of mind of the user. The processing unit includes a mobile application to facilitate the user to provide a drawing drawn by the user. The user provides the drawing by inputting a pre-drawn image, and/or drawing an image on a user interface by selection of one or more shapes and colors. Additionally, the mobile application facilitates the user to initiate identification of the state of mind by selection of an interpretation button.

In an embodiment, the processing unit includes a backend system to receive the provided drawing and/or user data while providing the image. The user data while providing the drawing corresponds to a user input of current feeling, one or more user actions, a video capturing actions of the user while providing the drawing, and/or a heatmap of the user while providing the drawing. Further, the current feeling includes a heavy feeling and a light feeling. Additionally, the one or more user actions include a sequence of actions of the user while providing the drawing, technique of drawing of the user, and/or decisiveness of the user when providing the drawing. Further, the backend system converts the received drawing into a machine-understandable format.

In an embodiment, the processing unit includes an interpretation system to pre-process the received drawing to identify one or more shapes and/or one or more colors in the received drawing. In one embodiment, the pre-processing includes applying image processing on the received drawing to identify one or more shapes, one or more colors, or a combination thereof. In another embodiment, the pre-processing includes identifying one or more codes associated with the selected one or more shapes and colors to draw the image.

Further, the interpretation system identifies the state of mind of the user based on the received user data, the identified one or more shapes, and/or the identified one or more colors by employing one or more Machine Learning (ML) models. Further, the one or more ML models are trained by annotating a plurality of drawings with known shapes and colors and attributing patterns of state of mind with combinations of colors and shapes. Upon identifying the state of mind of the user, the interpretation system determines one or more recommendations from a plurality of pre-stored recommendations based on the identified state of mind of the user. Thereafter, the mobile application renders the identified state of mind of the user and the determined one or more recommendations to the user.

An embodiment of the present disclosure includes a method for identifying the state of mind of the user. The method includes the steps of facilitating the user to provide a drawing drawn by the user. Upon providing the drawing, the method includes the steps of facilitating the user to initiate identification of the state of mind by selection of an interpretation button. Further, the method includes the steps of receiving the provided drawing and/or user data while providing the image and converting the received drawing into a machine-understandable format. Further, the method includes the steps of pre-processing the received drawing to identify one or more shapes and one or more colors in the received drawing.

Further, the method includes the steps of identifying the state of mind of the user based on the received user data, the identified one or more shapes, and the identified one or more colors by employing one or more Machine Learning (ML) models. Additionally, the method includes the steps of determining one or more recommendations from a plurality of pre-stored recommendations based on the identified state of mind of the user. Thereafter, the method includes the steps of rendering the identified state of mind of the user and/or the determined one or more recommendations to the user.

### BRIEF DESCRIPTION OF THE DRAWINGS

The present subject matter will now be described in detail with reference to the drawings, which are provided as illustrative examples of the subject matter to enable those skilled in the art to practice the subject matter. It will be noted that throughout the appended drawings, features are identified by reference numerals. Notably, the FIGUREs and examples are not meant to limit the scope of the present subject matter to a single embodiment, but other embodiments are possible by way of interchange of some or all of the described or illustrated elements and, further, wherein:

FIG. 1 illustrates an exemplary environment of a processing unit for identifying state of mind of a user, in accordance with an embodiment of the present disclosure;

FIGS. 2A and 2B illustrate process flow within the processing unit for identifying state of mind of the user, in accordance with an embodiment of the present disclosure;

FIG. 3 shows an exemplary method for identifying the state of mind of the user, in accordance with an embodiment of the present disclosure;

FIGS. 4A-4F show exemplary representation of Graphical User Interface (GUI) displayed to the user for determining the state of mind of the user, in accordance with an embodiment of the present disclosure; and

FIG. 5 illustrates an exemplary computer unit in which or with which embodiments of the present disclosure may be utilized.

### DETAILED DESCRIPTION OF THE EMBODIMENTS

The detailed description set forth below in connection with the appended drawings is intended as a description of exemplary embodiments in which the presently disclosed disclosure can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed disclosure. However, it will be apparent to those skilled in the art that the presently disclosed disclosure may be practiced without these specific details. In some instances, well-known structures and devices are shown in block diagram form in order to avoid obscuring the concepts of the presently disclosed disclosure.

Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and/or firmware.

Embodiments of the present disclosure may be provided as a computer program product, which may include a non-transitory, machine-readable storage medium tangibly embodying thereon instructions, which may be used to program the computer (or other electronic devices) to perform a process. The machine-readable medium may include, but is not limited to, fixed (hard) drives, semiconductor memories, such as Read Only Memories (ROMs), Programmable Read-Only Memories (PROMs), Random Access Memories (RAMs), Erasable PROMs (EPROMs), Electrically Erasable PROMs (EEPROMs), flash memory or other types of media/machine-readable medium suitable for storing electronic instructions (e.g., computer programming code, such as software or firmware).

Various methods described herein may be practiced by combining one or more non-transitory, machine-readable storage media containing the code according to the present disclosure with appropriate standard computer hardware to execute the code contained therein. An apparatus for practicing various embodiments of the present disclosure may involve one or more computers (or one or more processors within the single computer) and storage systems containing or having network access to a computer program(s) coded in accordance with various methods described herein, and the method steps of the disclosure could be accomplished by modules, routines, subroutines, or subparts of a computer program product.

The terms “connected” or “coupled” and related terms are used in an operational sense and are not necessarily limited to a direct connection or coupling. Thus, for example, two devices may be coupled directly, or via one or more intermediary media or devices. As another example, devices may be coupled in such a way that information can be passed therebetween, while not sharing any physical connection. Based on the disclosure provided herein, one of ordinary skill in the art will appreciate a variety of ways in which connection or coupling exists in accordance with the aforementioned definition.

If the specification states a component or feature “may,” “can,” “could,” or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

It will be appreciated by those of ordinary skill in the art that the diagrams, schematics, illustrations, and the like represent conceptual views or processes illustrating processing units and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular name.

Embodiments of the present disclosure relate to a method and processing unit for determining the state of mind of a user without any intervention from a therapist. Such determination of the state of mind of the user is performed through a color-mending process. The goal of the color mending process is to help the user build emotional literacy for identifying what emotions the user is feeling, understanding why the user is feeling that way, and facilitating the user to productively express emotions. The processing unit allows the user to color whatever they want, interpreting the coloring, and asking the user if they agree or disagree with the interpretation. Further, contemplating and articulating why they agree or disagree helps the user build a greater sense of self-trust, strengthens their metacognition, and helps the user learn emotions are just information.

The processing unit includes a mobile application to facilitate the user to provide a drawing drawn by the user. Further, the processing unit includes a backend system to receive the provided drawing and/or user data while providing the image. The backend system also converts the received drawing into a machine-understandable format. Further, the processing unit includes an interpretation system to pre-process the received drawing to identify one or more shapes and/or one or more colors in the received drawing. The interpretation system also identifies the state of mind of the user based on the received user data, the identified one or more shapes, and/or the identified one or more colors by employing one or more Machine Learning (ML) models. Thus, the state of mind is determined using an image drawn by the user using a user device. In real-time, the user may be prompted to draw an image using selected shapes and colors. The image may be provided as an input to the processing unit and analyzed to identify the state of mind, dynamically. The processing unit implements an AI/ML model to analyze the input and identify the state of mind. The processing unit performs pre-processing to extract details of shapes and colors used in the images drawn by the user. Further, the extracted shapes and colors are analyzed using pre-stored data to identify behavior characteristics and the state of mind of the user.

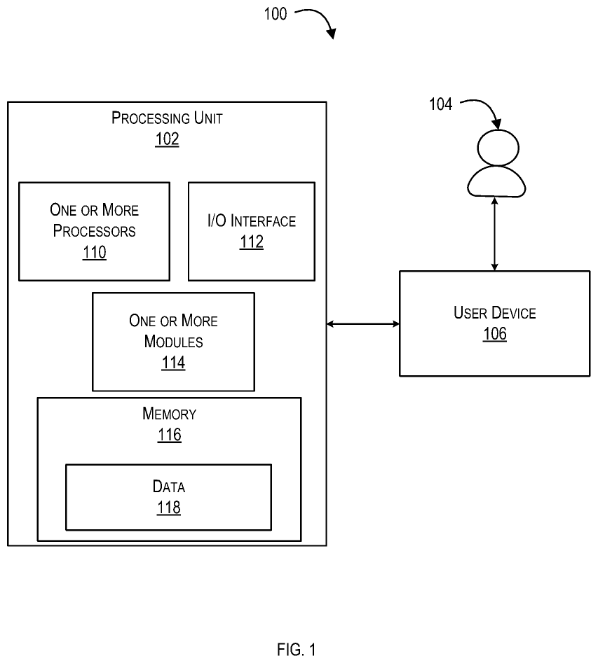

FIG. 1 illustrates an exemplary environment 100 of a processing unit 102 for identifying the state of mind of a user, in accordance with an embodiment of the present disclosure. The exemplary environment 100 comprises the processing unit 102 (may also be referred to as a system 102), and a user device 106 connecting the user 104 with the processing unit 102. The processing unit 102 may identify the state of mind of the user 104. The user 104 may use the user device 106 to provide input to the processing unit 102 and receive output from the processing unit 102. In an embodiment, the user device 106 may include, but is not limited to, a smartphone, a computer, a Personal Assistant Device (PDA), a tablet, a laptop, a desktop, and so on. The user device 106 may be any electronic device that facilitates the user 104 to provide input for analyzing and determining the state of mind and also recommend therapies based on the determined state of mind. In an embodiment, the processing unit 102 may be implemented in the user device 106 for identifying the state of mind. In an alternate embodiment, part of the processing unit 102 may be implemented in the user device 106 and other parts of the processing unit 102 may be implemented external to the user device 106 in the form of a server in communication with the user device 106. In an embodiment, said server may be a dedicated server or a cloud-based server that receives the input and provides the output in real-time to the user 104 via the user device 106. The processing unit 102 may include one or more processors 110, an Input/Output (I/O) interface 112, one or more modules 114, and a memory 116. In some non-limiting embodiments or aspects, the memory 116 may be communicatively coupled to one or more processors 110. The memory 116 stores instructions, executable by the one or more processors 110, which on execution, may cause the processing unit 102 to identify the state of mind of the user 104. In some non-limiting embodiments or aspects, the memory 116 may include data 118. The one or more modules 114 may perform the steps of the present disclosure using the data 118 to identify the state of mind. In some non-limiting embodiments or aspects, each of the one or more modules 114 may be a hardware unit, which may be outside the memory 116 and coupled with the processing unit 102. The I/O interface may be used to transmit and receive data that may be required to identify the state of mind. In some non-limiting embodiments or aspects, the processing unit 102 may be implemented in a variety of computing systems, such as a laptop computer, a desktop computer, a Personal Computer (PC), a notebook, a smartphone, a tablet, e-book readers, a server, a network server, a cloud server, and the like.

In an embodiment, the processing unit 102 may be implemented as a cloud-based server that communicates with the user device 106 via a communication network (not shown in the Figure). The communication network may include, without limitation, a direct interconnection, a Local Area Network (LAN), a Wide Area Network (WAN), a wireless network (e.g., using Wireless Application Protocol), the Internet, and the like. In an embodiment, the processing unit 102 may communicate with multiple user devices to determine the state of mind of multiple users. In such an embodiment, the processing unit 102 may communicate with each of the multiple user devices using a single communication network or a corresponding dedicated communication network.

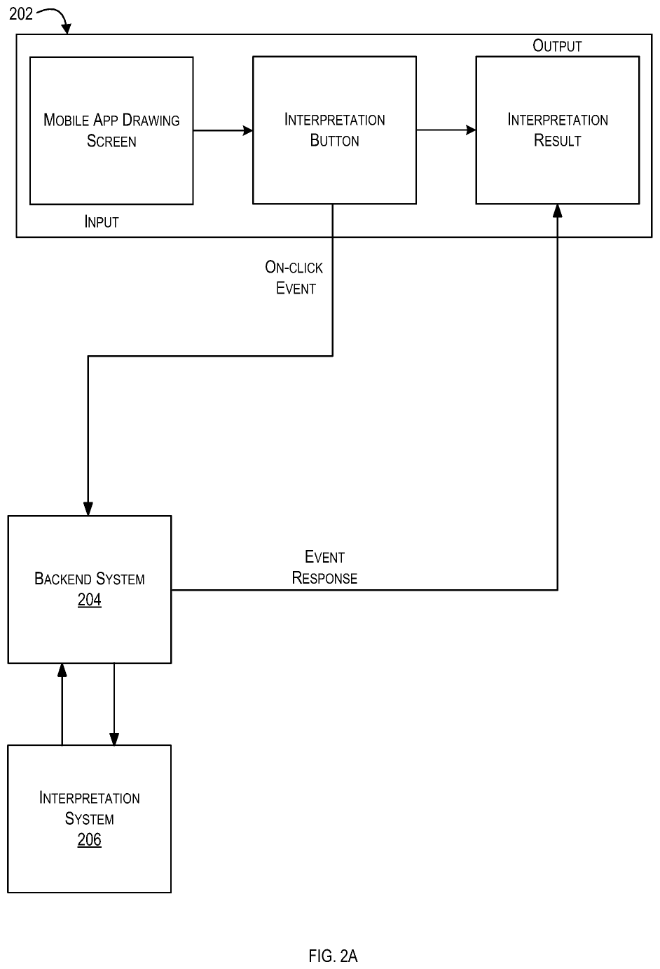

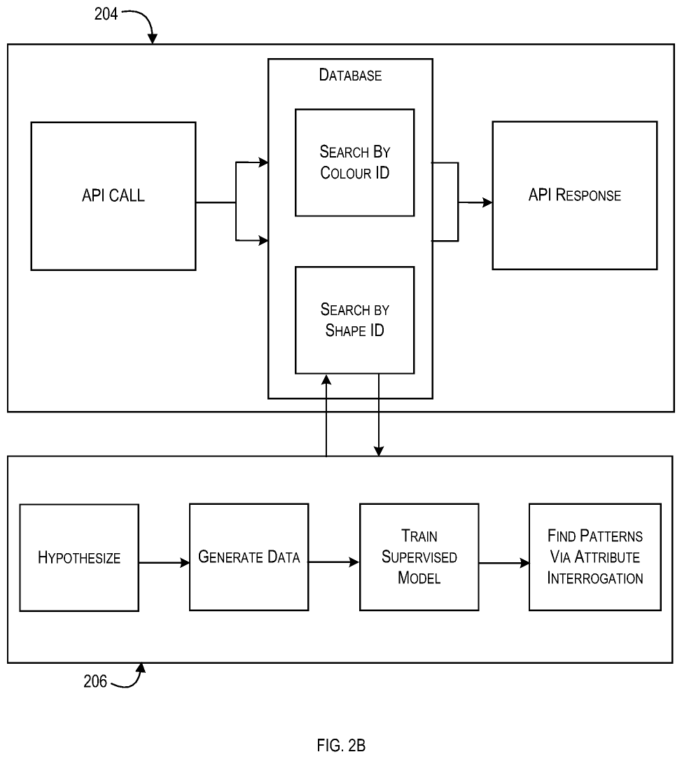

FIGS. 2A and 2B illustrate the process flow within the processing unit 102, in accordance with an embodiment of the present disclosure. In an embodiment, the processing unit 102 may operate to identify the state of mind using three components. The three components may include, but are not limited to, a mobile application 202, a backend system 204, and an interpretation system 206. As shown in FIG. 2A, the mobile application 202 may be used to receive the input from the user 104 and provide the output to the user 104. The mobile application 202 may broadly constitute a user interface having a mobile app drawing screen, an interpretation button, and a display of interpretation results. The mobile app drawing screen may enable the user 104 to draw desired images by selected colors. In an embodiment, the mobile application 202 may be aesthetically designed in a way that does not influence the mental state of the user 104, for example, the colors used in the designs may be black, grey, and white; and/or the design and copy within the mobile application 202 may be minimal, clean, and organized. In an embodiment, the mobile application 202 may act as a black canvas (such as a graph having x and y coordinates) for the user 104 to articulate the emotions that they may be feeling through drawing and typing. Further, it may be understood that the more coloring sessions a user completes within the mobile application 202, the more robust and personalized the mobile application 202 may become both in terms of the AI capabilities in interpreting the drawings, and the information a user types into the mobile application 202 that keeps a record of the user's mental state, metacognition, and emotional literacy growth over time. Furthermore, the coloring sessions may be recorded and the recorded coloring sessions may also be beneficial for sharing with third parties (such as a therapist, counselor, mental health professional, teacher, or parent/guardian) for additional support and guidance. Such third parties may keep a record of areas of concern/growth and determine what to address during third sessions with the user 104.

The image (such as a circle, flower, tree, or the like) drawn by the user 104 may be considered as the input to the backend system 204 and the interpretation system 206 for identifying the state of mind. Upon completion of drawing the image, the user 104 may select the interpretation button to initiate the analysis of the image. Selection of the interpretation button may initiate an on-click event for analyzing the drawn image. For the analysis, the image may be communicated with the backend system 204. The backend system 204 along with the interpretation system 206 may analyze the image to provide an event response. The event response is the output i.e., the interpretation results indicative of the state of mind of the user 104. In an embodiment, the mobile application 202 may monitor user actions when drawing the image on the mobile app drawing screen. The user actions may include, sequence of actions of the user 104 when drawing the image, technique of drawing of the user 104, decisiveness of the user 104 when drawing the image and so on. Along with the image, such monitored user actions may also be shared for analyzing and determining the state of mind of the user 104. In an embodiment, techniques known to a person skilled in the art may be implemented to monitor the user action. For example, session recordings may be performed and heatmaps may be created to monitor the user actions.

As shown in FIG. 2B, the backend system 204 along with the interpretation system 206 may operate to interpret the behavior and the state of mind of the user 104. In an embodiment, the backend system 204 and the interpretation system 206 may be implemented within a cloud server architecture for analyzing the image and providing the output to the user 104. When the interpretation button is selected by the user 104, the backend system 204 may start to operate for analyzing the input. The backend system 204 may include one or more units which support the operation of analysis. The one or more units include, but are not limited to, API call, database, and API response. When the interpretation button is selected by the user 104, the API call functions to call or invoke the AI-ML model and aid in the transmission of the input from the mobile application 202 to the backend system 204. The AI-ML model may be trained to annotate the drawing for the classification that it is part of. Further, during training, a picture is taken up as a data set and passed on to the model for further training. The backend system 204 may communicate with the mobile application 202 to receive the image. The image may be converted, by the mobile application 202, to a format that may be compatible for analysis i.e., a machine understandable format like electronic codes. In an embodiment, the input may be a video that captures the actions of the user 104 when drawing the image.

The AI/ML model is referred to as the interpretation system 206 in the FIGURES. In an embodiment, the AI/ML model may be created in Python, and API to access the AI/ML model may be created using Django. In an embodiment, the AI/ML model and API may work as a microservice with a main platform. Microservice architecture is recommended to avoid overloading the main server and gives the AI-ML model more processing power. In an embodiment, the AI/ML model may be used as a plug-and-play service. Moreover, even if a change in the logic of the AI/ML model is required, the main platform may remain intact from changes from a scalability perspective. The ML model may include a deep learning framework such as Convolutional Neural Network (CNN). In order to perform the color and shape detection process, the execution setup of the ML model may include installing Python on the user device, setting up the Python library and its dependencies, writing color and shape detection custom codes, and downloading the model file.

The interpretation system 206 may be pre-trained and further used in real-time for analyzing the received inputs. The interpretation system 206 includes hypothesis, generation of data, training supervised model, and finding patterns via attribution interrogation. In hypothesis, the AI/ML model is trained to correlate shapes and colors with human characteristics of behaviors or mental states. In an embodiment, the ML model may identify all colors used, for example, if someone draws a red square and a pink circle and then covers colors over both shapes using the color grey, then the ML model may be able to detect all three colors, not solely the grey colors. Data required for correlation may be pre-fed by the user 104. The generation of data is performed by annotating a plurality of images with known shapes and colors. In an embodiment, the correlated data and the generated data are used to train the supervised model to annotate for binary classifications and return a list of content categories that apply to the picture (drawing) date. Upon training the AI/ML model, patterns of state of mind are attributed with combinations of colors and shapes. By this process, the AI/ML model is trained to operate in real-time to analyze the attributes in the received image and identify the state of mind of the user 104. In real-time, the database with pre-stored attributes related to the shapes, colors and human characteristics behavior and state of mind is used to analyze the image and identify the state of mind of the user 104. In an embodiment, each of the colors and shapes may be associated with a unique ID. When the image is inputted, the image processing may be performed on the image to identify unique IDs associated with the image. The pre-stored attributes may be referred to as master set of data which help in analyzing the image. In an embodiment, the pre-stored attributes may be in form of look-up table which may be used to map the extracted shapes and colors with state of mind of the user 104. Exemplary representation of the look-up table with mapping between colors and shapes with state of mind or human characteristics is illustrated in Tables 1 and 2.

TABLE 1

| Color | Heavy Meanings | Light Meanings |
|---|---|---|
| RED | Anger, frustration, hate | Love, feeling connected to self and |
| | | others |
| ORANGE | Confusion, absorbing others' emotions, | Acceptance, understanding what's |
| | blocked creativity | yours vs. others, creativity |
| YELLOW | Judging yourself (or others), not feeling | Able to trust self (and others), joy, |
| | good or strong enough | high self-esteem |
| GREEN | Sadness, feeling abandoned, grief | Feelings of approval and love, |
| | | forgiveness and acceptance |
| YELLOW GREEN | Difficulty making decisions, feeling | Confidence in decisions, self-love |
| | worthless | |
| BLUE | Feeling like you can't express yourself, | Being able to express yourself, peace |
| | sadness | |
| PURPLE | Feeling alone, judging self (or others), | Feeling connected, trusting yourself |
| | overthinking things, mad at The | and others, peace with The |
| | Universe/Diety/authority | Universe/Diety/ authority |
| CLEAR/WHITE | Not wanting to be seen, pretending | Truth, light, intelligence, clarity, |
| | everything is okay | innocence, purity |
| PINK | Unexpressed love, blocking the flow of | Being open to giving and receiving |
| | love | love |
| BROWN | Feeling out of control, fear | Feeling in control of your life, feeling |
| | | safe, feeling worthy of comfort |
| BLACK | Fear, intense feelings, generational | Feeling calm and peace, ability to |
| | beliefs, blocking your learning | see what's yours and what's |
| | | someone else's, feeling connected |
| GRAY/SILVER | Confusion, being hard on yourself, | Ability to see someone else's |
| | overthinking | perspective, self-love |
| GOLD | Believing “I am bad,” unable to see | Self-love and forgiveness, high |
| | self-worth, being selfish | self-worth, feeling charitable |
| RAINBOW | Needing to make everything look | Being able to accept things as they |
| | better than it really is | are, peace, love, healing |
| CRYSTALS/ | Covering up negativity, working hard to | Letting your inner light shine, clarity, |
| GLOWING | appear spiritual | peace |

TABLE 2

| Shape | What it is a reflection of . . . |
|---|---|
| 3D Shapes | Your spiritual connection to self and the Universe |
| Airplane/Rocket | Your motivation and direction |
| Arrow | Bringing your attention to something |
| Bag | Your ambition |
| Ball | Self-worth and integration with self |
| Balloon | A reminder to be authentic and live in joy |
| Bat (animal) | How you approach obstacles |
| Bird | Your goals and dreams |
| Blob | How open you are to seeing something as it truly is |
| Blood | Generational belief or pattern |
| Boat | The arrival of news (typically fear around what it'll be) |
| Bone | How difficult it is to change something |
| Border | How you seek control, what you're using to control |
| Bowl | Your openness to receiving abundance |
| Box | Trying to conceal or reveal something |
| Bridge | Connections or transitions |
| Bubbles | Satisfaction with a specific situation |
| Butterfly | Transition and change |
| Cage | Feeling imprisoned, suppressed, or withheld |
| Candy | Love, joy, and fun |
| Car | Your approach to life |
| Castle | Your career |
| Cat | Your loyalty to yourself |
| Chair | Views on rest and relaxation |
| Chocolate | How sweet life feels |
| Circle | Feeling incomplete and/or uncertain |
| Clamp | Need to hold something together, or lock it tight |
| Clothing | How you present yourself to the world |
| Cloud | Feelings of depression or extreme sadness |
| Cube | Something that is blocking your joy |
| Cylinder | Your connection to the Universe |
| Diamond | Being stubborn/refusing to change |
| Dinosaur | An unresolved experience |
| Dirt | Lack of direction, emotional instability |
| Dog | Your intuition |
| Dynamite/Explosion | Suppressed emotions and/or words |
| Earth | The foundation of your beliefs |
| Elephant | Your prosperity and wisdom |
| Envelope | Your openness to receiving messages |
| Explosion/Dynamite | Bottling up emotions and/or words |
| Eye(s) | How open you are to seeing something as it truly is |
| Fabric | Emotional desires (typically around love) |
| Face | How you perceive yourself |
| Fence | Boundaries |
| Fire | How open you are to learning, how in control you feel |
| Fish | Personal growth |
| Flower | Your happiness |
| Foot | Your sense of stability |
| Frog | Personal changes |
| Fruit | Your spirituality |
| Glass | A transformation |
| Grass | Views on abundance and wealth |
| Hair | How strong and/or free you feel |
| Hand | Your belief in your power to do something |
| Hawk | Your ability to move forward |
| Haze/Mist/Overlay | Confusion or fear |
| Heart | The emotions you're currently feeling |
| Hexagon | Balance and harmony |
| Hook | The health of your attachments |
| Horse | Your health |
| House | What is happening internally for you |
| Kite | Your joy, hope, and dreams |
| Knife | Loss or change |
| Lady bug | Happiness and good luck |
| Lava | Old emotions |
| Leaf | Growth |
| Lightning Bolt/ Zig Zag | Something that needs your attention |
| Line | Limiting beliefs |
| Lips/Mouth | Feeling or being deceived |
| Liquid/Water | Control, fear, suppressed emotion |
| Mist/Overlay/Haze | Confusion or fear |
| Monkey | Curiosity or deceifulness |
| Monster | Bottled up emotions |
| Moon | Emotions and feelings (relating to feminine energy) |
| Mountain | Obstacles along/in your path |
| Mouth/Lips | Feeling or being deceived |
| Needle | Worries (especially in relationships) |
| Octagon | Repeating patterns or cycles |
| Oval | How balanced your feminine energy is |
| Overlay/Haze/Mist | Confusion or fear |
| Path | The path you're currently on, or ware wanting to be on |
| Pen/Pencil | How free you feel to reach your potential |
| Penguin | How you adapt to change |
| Pig | How you view yourself |
| Poop | How free you feel |
| Rabbit | Love and sexuality |
| Rain | How ready you are to release something |
| Rainbow | A turning point |
| Rectangle | How stable you feel |
| Reptile | your instincts |
| Ring | Commitment to a person/self/goal/desire |
| Rocket/Airplane | Your motivation and direction |
| Rock | Strength and ability |
| Rod | Ease in communication (often tied to generational patterns) |
| Rope | How complex/simple things feel |
| Shadow | What you are rejecting/accepting |
| Shark | How balanced the emotions your feeling are |
| Skull | Things coming to an end |
| Slime | Your ability to trust |
| Sludge | How stagnant/free you feel |
| Snake | Manipulation (typically fueled by fear) |
| Spider | Something you need to pay attention to |
| Spike | Anger (the more points the greater the anger) |
| Spiral/Swirl | Need for change, using sarcasm to cope |
| Square | Your loyalty to yourself |
| Stairs | Your progression through life |
| Star | How you feel about your destiny or goals |
| String/Yarn | How connected and creative you feel |
| Stripe | Your hope that someone/something will change |
| Sun | Entering into a new experience |
| Swirl/Spiral | Need for change, using sarcasm to cope |
| Sword | Your personal power or another's power |
| Teeth | How you fit in, your patience |
| Tornado | Fear, anxiety, or drama |
| Tractor | Your work |
| Train | Stability and predictability |
| Tree | Generational patterns affecting your growth |
| Triangle | Your connection with your spiritual self |
| Umbrella | Your safety |
| Vomit | Wanting to find a better way to connect to life/the Universe |
| Water/Liquid | Control, fear, suppressed emotion |
| Wave | The emotions you're feeling |
| Whale | Your relationship to self, family, friends |
| Wing(s) | Your energy and strength |
| X | Shame (“I am bad”), or your satisfaction |
| Yarn/String | How connected and creative you feel |
| Zig Zag/Lightning Bolt | Something that needs your attention |

In an embodiment, the backend system 204 and the interpretation system 206 may be implemented in separate servers in the cloud server architecture. Thus, the mobile application 202 communicates with the interpretation system 206 via the backend system 204 and not directly. The state of mind identified may be pushed to the mobile application 202 via the API response in form of the event response. In the mobile application 202, the interpretation result is displayed to the user 104. In an embodiment, the mobile application 202 may be developed using Flutter (a cross-platform), the backend system 204 may be created using PHP (Laravel Framework) and the database may be created using My SQL.

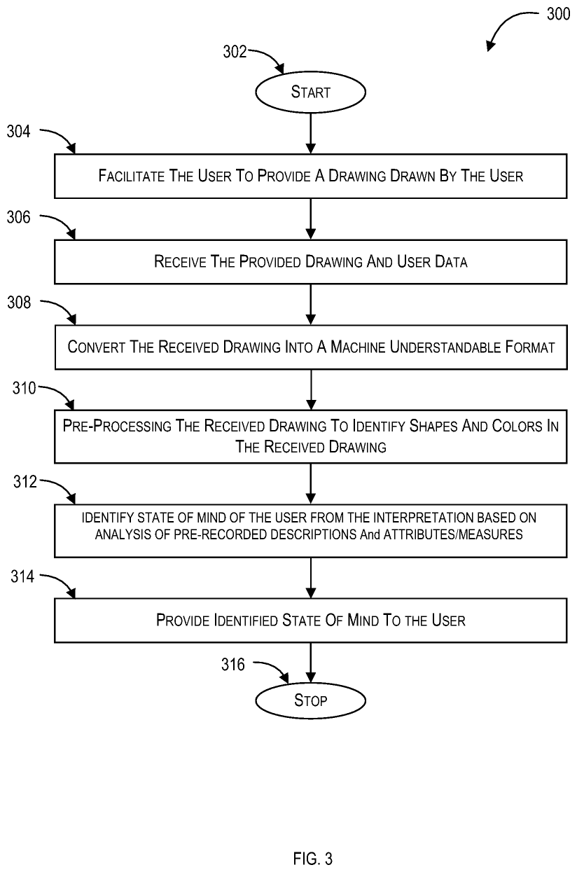

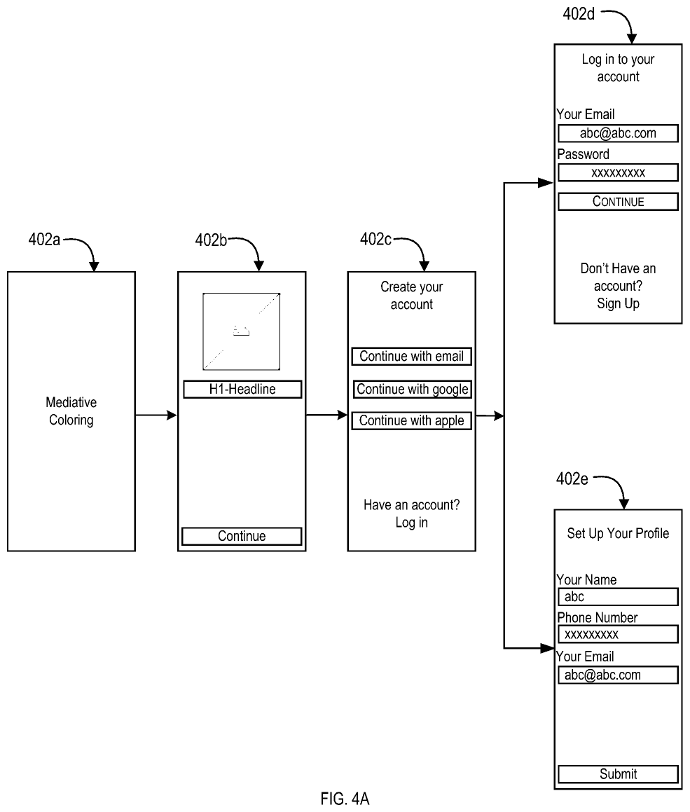

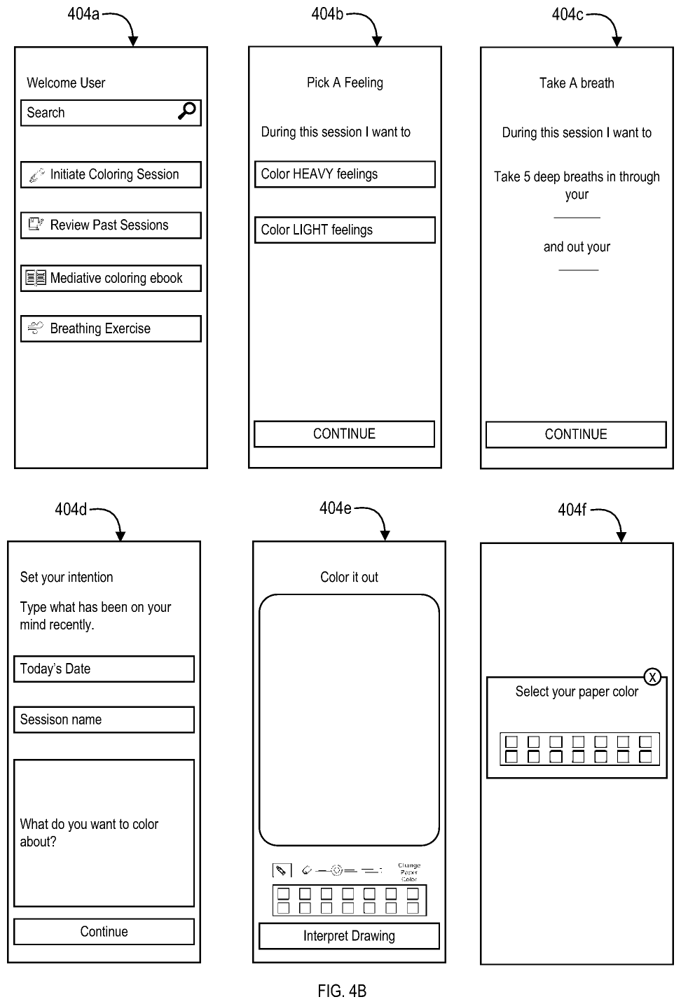

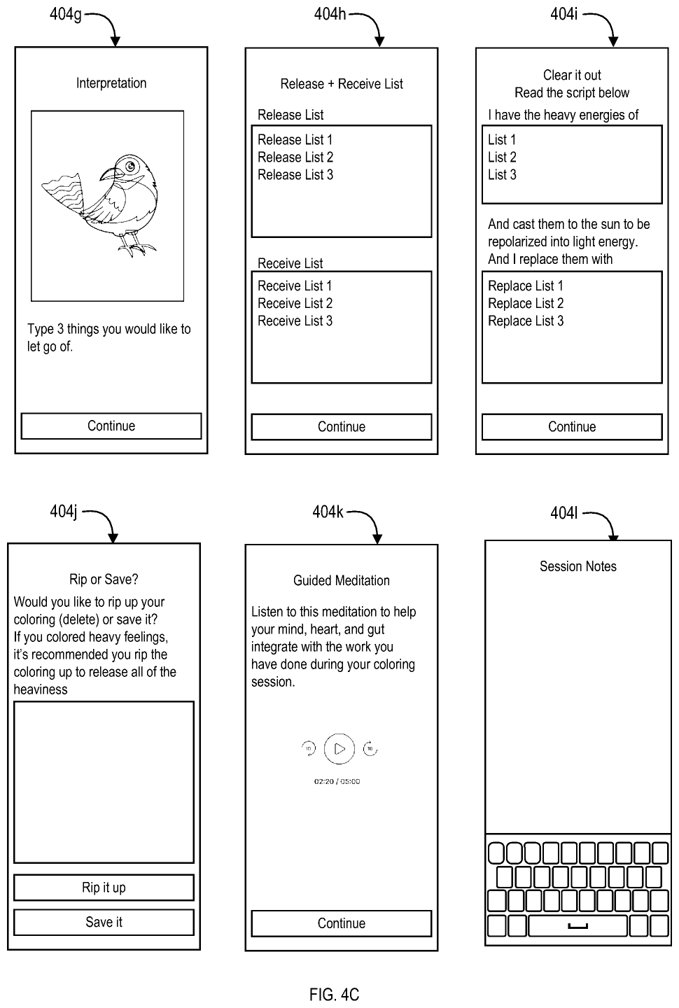

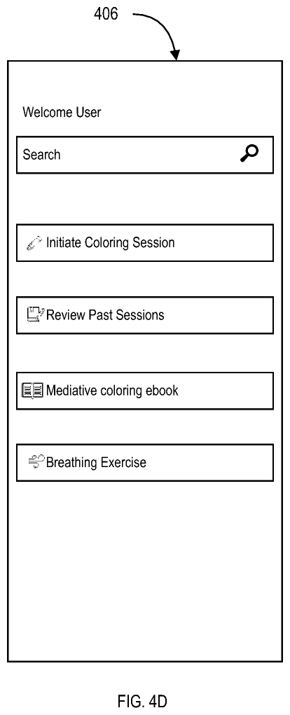

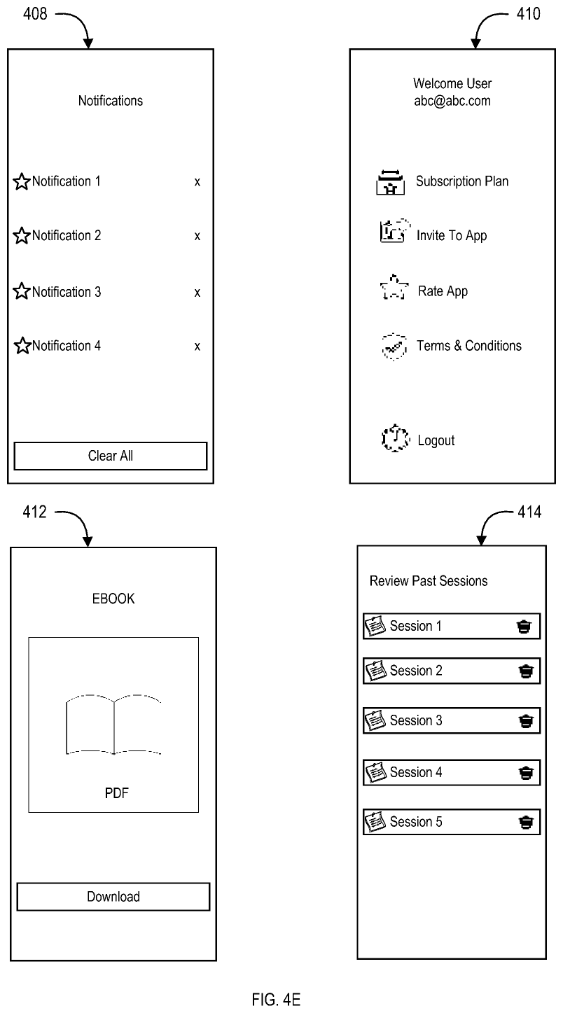

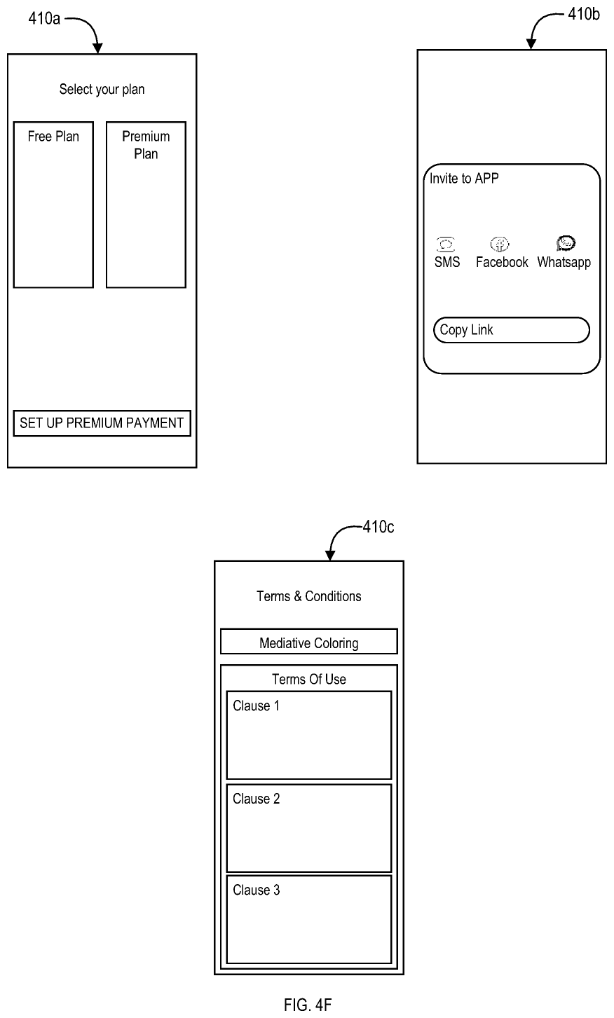

FIG. 3 shows an exemplary method 300 for the state of mind of the user 104, in accordance with an embodiment of the present disclosure. FIGS. 4A-4F show exemplary representation of Graphical User Interface (GUI) displayed to the user 104 for determining the state of mind of the user 104, in accordance with an embodiment of the present disclosure. For the sake of brevity, FIGS. 3 and 4A-4F have been explained together. The method starts at step 302.

In an embodiment, the exemplary method 300 for identifying the state of mind of the user 104 includes steps coded in the form of executable instructions to be executed by the processing unit 102. In real-time, the user 104 may be provided within the mobile application 202. The process of onboarding the user 104 using the mobile application 202 is illustrated in FIG. 4A. In an embodiment, the user 104 may access the application via a web browser. Interfaces 402a and 402b show the initial display of loading the mobile application 202 in the user device 106. The user 104 may be provided to create an account as shown in interface 402c, using an existing account. Further, if the user 104 has an existing account, the user 104 may merely sign-in to the mobile application 202 as shown in interface 402d or the user 104 may sign-up with the mobile application 202 as shown in interface 402e. Upon signing-in with the mobile application 202, the process of identifying the state of mind of the user 104 may be initiated by prompting the user 104 to input an image for analysis. FIGS. 4B and 4C illustrate exemplary displays of the interface of the user device 106 during the process of identifying the state of mind of the user 104.

At step 304, the processing unit 102 may facilitate the user to provide a drawing drawn by the user 104. Upon logging in the user 104 may be prompted with multiple options as shown in interface 404a. The options may include initiating the coloring session, reviewing past sessions of the coloring, refer meditative coloring e-book to understand the working of the mobile application 202, or initiate a therapy session like breathing exercises. In an embodiment, when the user 104 selects to initiate the coloring session, interface 404b may be displayed to the user 104. The user 104 may be prompted to choose what type of feeling they would like to color. This feeling could be their current feeling, or a past feeling they want to learn more about. The feeling may be a heavy feeling or a light feeling. In case the user 104 selected feelings like frustrated, sad, or unhappy, the feeling may be chosen to be the heavy feeling. In case the user 104 selected feelings like happy or calm, the feeling may be chosen to be the light feeling. Upon choosing the feeling, the user 104 may be displayed with interface 404c in an attempt to prepare the user 104 for a coloring session. In case the user 104 is frustrated, sad, or not happy, the current feeling may be chosen to be the heavy feeling. In case the user 104 is happy or calm, the current feeling may be chosen to be the light feeling. Upon choosing the current feeling, the user 104 may be displayed with interface 404c in attempt to prepare the user 104 for a coloring session. In an embodiment, the user 104 may be given an option to set an intention for taking the coloring session as shown in interface 404d. The user 104 may be provided with an option to manually input his intentions and a few other details that he may wish to record before the commencement of the session. In an embodiment, the intentions entered by the user 104 may be merely used for user's reference when current session is viewed as past session in future. Alternatively, the intentions input by the user 104 may be used by the processing unit 102 to analyze the image and identify the state of mind accurately. Upon receiving the intentions, a drawings screen may be displayed in the user device 106 as shown in interface 404e enabling the user to draw an image. In an embodiment, the user 104 may be provided with multiple options relating to coloring. For example, as shown in interface 404f, the user 104 may be able to choose paper color for the drawing screen. In another embodiment, the multiple options may include changing weight of the lines used for drawings, option to choose templates of shapes, resize the images drawn and so on. The image drawn by the user 104 is considered as the input received by the processing unit 102 for the analysis. Next, at step 306, the provided drawing and the user data while providing the image may be received. Upon receiving, the received drawings may be converted into a machine understandable format, at step 308.

At step 310, the received drawing may be pre-processed to identify one or more shapes and/or one or more colors in the received drawing. The received drawing may be parsed to extract the shapes and colors used in the image. In one embodiment, the pre-processing may include applying image processing on the received drawing to identify the or more shapes and/or one or more colors. In another embodiment, the pre-processing may include identifying one or more codes associated with the selected one or more shapes and colors to draw the image.

At step 312, the state of mind of the user 104 may be identified based on the received user data, the identified one or more shapes, and the identified one or more colors by employing one or more Machine Learning (ML) models. For the identification, the extracted shapes and colors may be mapped with pre-fed human characteristics or state of mind using look-up tables illustrated in Tables 1 and 2. Using the mapping, the final state of mind of the user 104 may be identified by the processing unit 102. For example, consider the image in interface 404g provided by the user 104. The image may be parsed to identify the image to be related to a bird. Consider the color used in the image is yellow and the current feeling is selected to be the heavy feeling. Birds are related to goals and dreams. The heavy feeling of yellow color relates to having a fear of judgement. By analysis of such data, the state of mind of the user 104 may be identified to be that the user 104 possesses fear of judgement of other people on his goals or dreams.

At step 314, the identified state of mind of the user 104 may be provided to the user 104. Along with merely displaying the state of mind to the user 104, the processing unit 102 may guide the user 104 to overcome negative state of mind. For example, in an embodiment, as shown in interfaces 404h, 404i, and 404j, the processing unit 102 may provide remedy to overcome the heavy feeling. In an embodiment, the processing unit 102 may form a release list and receive list including feelings that are to be released and feelings that are to be received by the user 104 based on the identified state of mind. In an embodiment, such feelings may be provided to the user 104 in the form of affirmations. In another embodiment, the user 104 may be prompted to rip off the negative feeling on the screen and save the positive feelings. In an embodiment, guided meditation may be provided to the user 104 as shown in interface 404k. At the end, the user 104 may be prompted to include personal notes on the current session as shown in the interface 404l.

The mobile application 202 may provide a few additional options to the user 104 as shown in interface 406 of FIG. 4D. Some of them are illustrated in FIGS. 4E and 4F. The user 104 may be notified on the identified state of mind as shown in interface 408. Other notifications may relate to reminding the user 104 to take proper remedies to overcome the negative state of mind. Other options may include to avail a plan to take-up coloring sessions, invite friends to use the application, rate the application and so on as shown in interface 410. The user 104 may be able to take-up multiple coloring sessions by availing a suitable plan, as shown in interface 410a in FIG. 4F. Invitation to use the application may be initiated by the user 104 as shown in interface 410b in FIG. 4F. Terms and conditions related to the application may be viewed as shown in interface 410c in FIG. 4F. Guide on usage of the mobile application 202 may be viewed or downloaded as shown in interface 412 in FIG. 4E. Past sessions may be viewed by the user 104 as shown in interface 414 in FIG. 4E. The method ends at step 316.

As illustrated in FIG. 3, the method 300 may include one or more steps for executing processes in the processing unit 102. The method 300 may be described in the general context of computer-executable instructions. Generally, computer-executable instructions can include routines, programs, objects, components, data structures, procedures, modules, and functions, which perform particular functions or implement particular abstract data types.

The order in which steps in the method 300 are described may not be intended to be construed as a limitation, and any number of the described method steps can be combined in any order to implement the method. Additionally, individual steps may be deleted from the methods without departing from the scope of the subject matter described herein. Furthermore, the method can be implemented in any suitable hardware, software, firmware, or combination thereof.

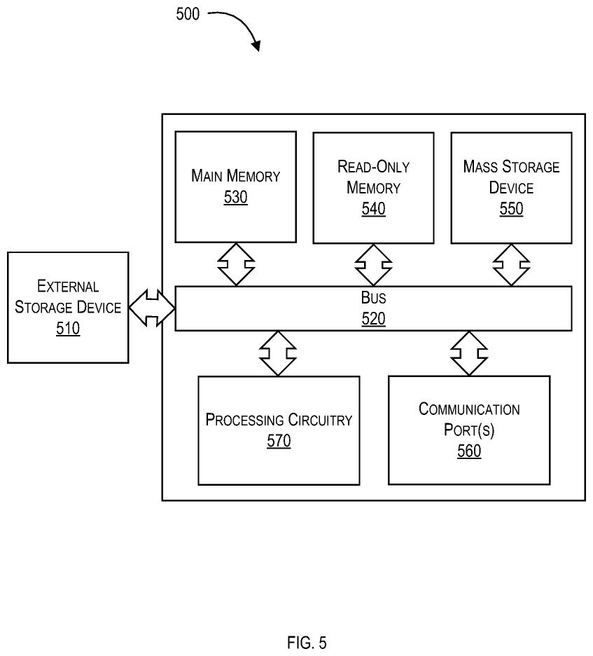

FIG. 5 illustrates an exemplary computer system in which or with which embodiments of the present disclosure may be utilized. Depending upon the particular implementation, the various process and decision blocks described above may be performed by hardware components, embodied in machine-executable instructions, which may be used to cause a general-purpose or special-purpose processor programmed with the instructions to perform the steps, or the steps may be performed by a combination of hardware, software and/or firmware. As shown in FIG. 5, the computer system 500 includes an external storage device 510, bus 520, main memory 530, read-only memory 540, mass storage device 550, communication port(s) 560, and processing circuitry 570.

Those skilled in the art will appreciate that the computer system 500 may include more than one processing circuitry 570 and one or more communication ports 560. The processing circuitry 570 should be understood to mean circuitry based on one or more microprocessors, microcontrollers, digital signal processors, programmable logic devices, Field-Programmable Gate Arrays (FPGAs), Application-Specific Integrated Circuits (ASICs), etc., and may include a multi-core processor (e.g., dual-core, quad-core, Hexa-core, or any suitable number of cores) or supercomputer. In some embodiments, the processing circuitry 570 is distributed across multiple separate processors or processing units, for example, multiple of the same type of processing units (e.g., two Intel Core i7 processors) or multiple different processors (e.g., an Intel Core i5 processor and an Intel Core i7 processor). Examples of the processing circuitry 570 include, but are not limited to, an Intel® Itanium® or Itanium 2 processor(s), AMD® Opteron® or Athlon MP® processor(s), Motorola® lines of processors, System on Chip (SoC) processors, or other future processors. The processing circuitry 570 may include various modules associated with embodiments of the present disclosure.

The communication port 560 may include a cable modem, Integrated Services Digital Network (ISDN) modem, a Digital Subscriber Line (DSL) modem, a telephone modem, an Ethernet card, or a wireless modem for communications with other equipment, or any other suitable communications circuitry. Such communications may involve the Internet or any other suitable communications networks or paths. In addition, communications circuitry may include circuitry that enables peer-to-peer communication of electronic devices or communication of electronic devices in locations remote from each other. The communication port 560 may be any RS-232 port for use with a modem-based dialup connection, a 10/100 Ethernet port, a Gigabit, or a 10 Gigabit port using copper or fiber, a serial port, a parallel port, or other existing or future ports. The communication port 560 may be chosen depending on a network, such as a Local Area Network (LAN), Wide Area Network (WAN), or any network to which the computer system 500 may be connected.

The main memory 530 may include Random Access Memory (RAM) or any other dynamic storage device commonly known in the art. Read-only memory (ROM) 540 may be any static storage device(s), e.g., but not limited to, a Programmable Read-Only Memory (PROM) chips for storing static information, e.g., start-up or BIOS instructions for the processing circuitry 570.

The mass storage device 550 may be an electronic storage device. As referred to herein, the phrase “electronic storage device” or “storage device” should be understood to mean any device for storing electronic data, computer software, or firmware, such as random-access memory, read-only memory, hard drives, optical drives, Digital Video Disc (DVD) recorders, Compact Disc (CD) recorders, BLU-RAY disc (BD) recorders, BLU-RAY 3D disc recorders, Digital Video Recorders (DVRs, sometimes called a personal video recorder or PVRs), solid-state devices, quantum storage devices, gaming consoles, gaming media, or any other suitable fixed or removable storage devices, and/or any combination of the same. Nonvolatile memory may also be used (e.g., to launch a boot-up routine and other instructions). Cloud-based storage may be used to supplement the main memory 530. The mass storage device 550 may be any current or future mass storage solution, which may be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firmware interfaces), e.g., those available from Seagate (e.g., the Seagate Barracuda 7200 family) or Hitachi (e.g., the Hitachi Deskstar 7K1000), one or more optical discs, Redundant Array of Independent Disks (RAID) storage, e.g., an array of disks (e.g., SATA arrays), available from various vendors including Dot Hill Systems Corp., LaCie, Nexsan Technologies, Inc. and Enhance Technology, Inc.

The bus 520 communicatively couples the processing circuitry 570 with the other memory, storage, and communication blocks. The bus 520 may be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects processing circuitry 570 to the software system.

Optionally, operator and administrative interfaces, e.g., a display, keyboard, and a cursor control device, may also be coupled to the bus 520 to support direct operator interaction with the computer system 500. Other operator and administrative interfaces may be provided through network connections connected through the communication port(s) 560. The external storage device 510 may be any kind of external hard drives, floppy drives, IOMEGA® Zip Drives, Compact Disc-Read-Only Memory (CD-ROM), Compact Disc-Re-Writable (CD-RW), Digital Video Disk-Read Only Memory (DVD-ROM). The components described above are meant only to exemplify various possibilities. In no way should the aforementioned exemplary computer system limit the scope of the present disclosure.

The computer system 500 may be accessed through a user interface. The user interface application may be implemented using any suitable architecture. For example, it may be a stand-alone application wholly implemented on the computer system 500. The user interfaces application and/or any instructions for performing any of the embodiments discussed herein may be encoded on computer-readable media. Computer-readable media includes any media capable of storing data. In some embodiments, the user interface application is client-server-based. Data for use by a thick or thin client implemented on electronic device computer system 500 is retrieved on-demand by issuing requests to a server remote to the computer system 500. For example, computer system 500 may receive inputs from the user via an input interface and transmit those inputs to the remote server for processing and generating the corresponding outputs. The generated output is then transmitted to the computer system 500 for presentation to the user.

While embodiments of the present disclosure have been illustrated and described, it will be clear that the disclosure is not limited to these embodiments only. Numerous modifications, changes, variations, substitutions, and equivalents, will be apparent to those skilled in the art without departing from the spirit and scope of the disclosure, as described in the claims.

Thus, it will be appreciated by those of ordinary skill in the art that the diagrams, schematics, illustrations, and the like represent conceptual views or processes illustrating processing units and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular name.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of this document, terms “coupled to” and “coupled with” are also used euphemistically to mean “communicatively coupled with” over a network, where two or more devices are able to exchange data with each other over the network, possibly via one or more intermediary device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded to the widest scope consistent with the principles and novel features disclosed herein. It is contemplated that additional embodiments are within the spirit and true scope of the disclosed subject matter.

What is claimed:

1. A processing unit for identifying state of mind of a user, the processing unit comprising:
a mobile application to facilitate the user to provide a drawing drawn by the user;
a backend system to:
receive at least one of: the provided drawing and user data while providing the image; and
convert the received drawing into a machine understandable format;
an interpretation system to:
pre-process the received drawing to identify at least one of: one or more shapes and one or more colors in the received drawing; and
identify the state of mind of the user based on at least one of: the received user data, the identified one or more shapes, and the identified one or more colors by employing one or more Machine Learning (ML) models.

2. The processing unit of claim 1, wherein the user provides the drawing by at least one of: inputting a pre-drawn image, and drawing an image on a user interface by selection of one or more shapes and colors.

3. The processing unit of claim 2, wherein the pre-processing includes applying image processing on the received drawing to identify at least one of: one or more shapes and one or more colors.

4. The processing unit of claim 2, wherein the pre-processing includes identifying one or more codes associated with the selected one or more shapes and colors to draw the image.

5. The processing unit of claim 1, wherein the user data while providing the drawing corresponds to at least one of: a user input of current feeling, one or more user actions, a video capturing actions of the user while providing the drawing, and a heatmap of the user while providing the drawing.

6. The processing unit of claim 5, wherein the current feeling includes at least one of: a heavy feeling and a light feeling.

7. The processing unit of claim 5, wherein the one or more user actions include at least one of: sequence of actions of the user while providing the drawing, technique of drawing of the user, and decisiveness of the user when providing the drawing.

8. The processing unit of claim 1, wherein the mobile application facilitates the user to initiate identification of the state of mind by selection of an interpretation button.

9. The processing unit of claim 1, wherein the one or more ML models are trained by:
annotating a plurality of drawing with known shapes and colors; and
attributing patterns of state of mind with combinations of colors and shapes.

10. The processing unit of claim 1, wherein the interpretation system further determines one or more recommendations from a plurality of pre-stored recommendation based on the identified state of mind of the user.

11. The processing unit of claim 10, wherein the mobile application further renders at least one of: the identified state of mind of the user and the determined one or more recommendations to the user.

12. A method for identifying state of mind of a user, the method comprising:
facilitating the user to provide a drawing drawn by the user;
receiving at least one of: the provided drawing and user data while providing the image;
converting the received drawing into a machine understandable format;
pre-processing the received drawing to identify at least one of: one or more shapes and one or more colors in the received drawing; and
identifying the state of mind of the user based on at least one of: the received user data, the identified one or more shapes, and the identified one or more colors by employing one or more Machine Learning (ML) models.

13. The method of claim 12, wherein the user provides the drawing by at least one of: inputting a pre-drawn image, and drawing an image on a user interface by selection of one or more shapes and colors.

14. The method of claim 13, wherein the pre-processing includes at least one of: applying image processing on the received drawing to identify at least one of: one or more shapes and one or more colors, and identifying one or more codes associated with the selected one or more shapes and colors to draw the image.

15. The method of claim 12, wherein the user data while providing the drawing corresponds to at least one of: a user input of current feeling, one or more user actions, a video capturing actions of the user while providing the drawing, and a heatmap of the user while providing the drawing.

16. The method of claim 15,
wherein the current feeling includes at least one of: a heavy feeling and a light feeling; and
wherein the one or more user actions include at least one of: sequence of actions of the user while providing the drawing, technique of drawing of the user, and decisiveness of the user when providing the drawing.

17. The method of claim 12, further comprises facilitating the user to initiate identification of the state of mind by selection of an interpretation button.

18. The method of claim 12, wherein the one or more ML models are trained by:
annotating a plurality of drawing with known shapes and colors; and
attributing patterns of state of mind with combinations of colors and shapes.

19. The method of claim 12, further comprises determining one or more recommendations from a plurality of pre-stored recommendation based on the identified state of mind of the user.

20. The method of claim 19, further comprises rendering at least one of: the identified state of mind of the user and the determined one or more recommendations to the user.
